(19) United States
(12) Patent Application Publication
(10) Pub. No.: US 2010/0249531 A1
(43) Pub. Date: Sep. 30, 2010

(54) MEDICAL HEALTH INFORMATION SYSTEM

(76) Inventors: Alaina B. Hanlon, Waltham, MA (US); Peter Connolly, Attleboro, MA (US); Steven K. Grinspoon, Weston, MA (US)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/727,728

(22) Filed: Jun. 21, 2010

Related U.S. Application Data
(60) Provisional application No. 61/161,672, filed on Mar. 19, 2009.
(60) Provisional application No. 61/254,428, filed on Oct. 23, 2009.

(51) Int. Cl. A61B 5/00; G06Q 50/00

(52) U.S. Cl. 600/300; 705/2

(57) ABSTRACT

A system and method determine and report a potential for development by a patient of disease and adverse health conditions. The method includes receiving patient phenotype data. The phenotype data can include, but is not limited to, biometric data specific to the patient, medical claims data specific to the patient, and organizational data specific to an organization to which the patient belongs. One or more predictive models are generated using one or more algorithms executing on at least one processor of a computing apparatus. The one or more predictive models determine and indicate the potential for development by the patient of disease and adverse health conditions, and output the potential to a user.

### RELATED APPLICATION

This application claims priority to, and the benefit of, co-pending U.S. Provisional Application 61/161,672, filed Mar. 19, 2009, and co-pending U.S. Provisional Application 61/254,428, filed Oct. 23, 2009, for all subject matter disclosed. The disclosures of said provisional applications are hereby incorporated by reference herein in their entirety.

### FIELD OF THE INVENTION

The present invention relates a system and method for providing personalized medical health care information to patients or user seeking such information.

### BACKGROUND OF THE INVENTION

Conventional health and wellness management programs may include Health Risk Assessments, biometric screening, health management tools, coaching, consulting, and/or reporting. However, such programs do not offer in-depth risk prediction. Likewise, they do not generally provide tools for improved management of modifiable risk factors. Conventional programs are additionally limited in customization of Health Risk Assessments and Reports.

### SUMMARY

There is a need for a health and wellness management platform provided in a computing environment that provides tools for improved management of potential for development by the patient of disease and adverse health conditions. The present invention is directed toward further solutions to address this need, in addition to having other desirable characteristics.

In accordance with one example embodiment of the present invention, a computer implemented method includes receiving patient data including one or more of phenotype data specific to a patient, biometric data specific to the patient, medical claims data specific to the patient, and organizational data specific to an organization to which the patient belongs. One or more predictive models are generated based on the patient data, using one or more algorithms executing on at least one processor of a computing apparatus, the one or more predictive models determining and indicating potential for development by the patient of disease and adverse health conditions. The potential for development by the patient of disease and adverse health conditions is output to the user.

In accordance with aspects of the present invention, the method can further include periodically automatically updating the patient data when new information is provided. The one or more predictive models can be periodically automatically updated to indicate the potential for development by the patient of disease and adverse health conditions by modifying the one or more algorithms.

In accordance with further aspects of the present invention, the phenotype data specific to the patient can include one or more data fields of the group of data fields comprising height, weight, waist circumference, biometric data, smoking frequency, alcohol consumption, lifestyle data, emotional data, and behavioral data. The biometric data specific to the patient can include one or more data fields of the group of data fields comprising total cholesterol, HDL cholesterol, LDL cholesterol, triglycerides, fasting glucose, hemoglobin A1c, ALT liver enzyme, C-Reactive Protein, and Complete Blood Count. The medical claims data specific to the patient can include one or more data fields of the group of data fields comprising health insurance claims for medical procedures, prescription medication cost, and doctor visit fees. The organizational data specific to an organization to which the patient belongs can include one or more data fields of the group of data fields comprising current health by condition, health risks by condition, productivity, absenteeism, lost time, predictive modeling, medical claims analysis, program eligibility, program participation, direct medical cost analysis, indirect medical cost analysis, and return on investment. The one or more algorithms can include one or more algorithms of the group of algorithms comprising coronary heart disease models, blood pressure models, cholesterol models, osteoporosis models, visceral fat models, diabetes models, metabolic syndrome models, and depression models.

In accordance with further aspects of the present invention, the step of outputting the potential for development by the patient of disease and adverse health conditions can include displaying via a user interface an indication of the potential. The method can further include providing access through the user interface to one or more of social networks, advertisements, and electronic communication tools. The method can further include sending out an automatic notification when a change to data or the one or more predictive models alters the indication of the potential for development by the patient of disease and adverse health condition.

In accordance with one embodiment of the present invention, in a networked computer environment, a system is provided that includes a storage device storing patient data comprising one or more of phenotype data specific to a patient, biometric data specific to the patient, medical claims data specific to the patient, and organizational data specific to an organization to which the patient belongs. At least one processor can be provided with executable instructions for generating one or more predictive models, using one or more algorithms executing on the at least one processor, the one or more predictive models determining and indicating a potential for development by the patient of disease and adverse health conditions. An output mechanism can be configured to output the potential for development by the patient of disease and adverse health conditions.

In accordance with one example embodiment of the present invention, a method can include providing patient data comprising one or more of phenotype data specific to a patient, biometric data specific to the patient, medical claims data specific to the patient, and organizational data specific to an organization to which the patient belongs, to a system executing a predictive model. The method can further include receiving an indication of a potential for development by the patient of disease and adverse health conditions generated by one or more predictive models, using one or more algorithms executing on at least one processor of a computing apparatus.

### BRIEF DESCRIPTION OF THE DRAWINGS

The present invention will become better understood with reference to the following description and accompanying drawings, wherein:

FIG. 1 is a networked computer environment for implementation of the present system and method, according to one aspect of the present invention;

FIG. 2 is a computing environment and computer apparatus operating within the networked computer environment for implementation of the present system and method, according to one aspect of the present invention;

FIG. 3 is a screenshot of a homepage user interface, according to one aspect of the present invention;

FIG. 4 is a screenshot of a another user interface with an advertising component, according to one aspect of the present invention;

FIG. 5 is a screenshot of another user interface with an advertising component, according to one aspect of the present invention; and

FIG. 6 is a diagrammatic illustration depicting a process for determining adverse health conditions considering a number of different factors, in accordance with one example embodiment of the present invention.

### DETAILED DESCRIPTION

The medical health system and method of the present invention as described herein provides a fully integrated health management and productivity solution built on the science of phenotyping. A genotype is conventionally defined as a trait or set of traits of an individual as dictated and determined by their genetic makeup. A phenotype is conventionally defined as the appearance, or expression of a trait, of an individual, resulting from the interaction of the genotype with the particular environment of the individual. Phenotyping is a methodology that looks at the environmental interactions, including lifestyle, behaviors, and environment, also considering genetics, to assess an individual's current and future health. The system and method of the present invention provide a platform that enable such activities as health risk and productivity assessments, biometric screening—including at home screening, customizable reports, coaching, education, tools that help drive engagement and compliance such as multi-channel communications and incentives, and consulting services to help define, implement, and manage health and wellness initiatives. The system and method reveals chronic diseases and other adverse health conditions before their onset and offers features and services to prevent, manage, and/or control health risks and conditions.

In accordance with one example embodiment of the present invention, the system and method provided herein uses phenotype and/or genotype characteristics of patients to provide customized medical health information.

Patients provide detailed information, such as through completion of a medical questionnaire, from which their phenotype can be determined. The medical questionnaire can include questions on a patient's basic physiological measurements (height, weight, waist circumference, etc.) as well as questions on the patient's medical history including medications used, illnesses, etc. The medical questionnaire can be an online form that can be similar to forms for collecting information in conventional medical predictor tools.

The system of the present invention can provide patients (perhaps with a registration prerequisite) with access to pharmaceutical and medical information that is specific to their health needs and their health risks by logging into a customized online health management portal.

The system also allows pharmaceutical and other medical companies to more efficiently and effectively target advertising to consumers. Pharmaceutical and other medical companies can pay to advertise relevant drugs, treatments, etc. to targeted patients. The advertisements are targeted to people with particular phenotypes and/or genotypes. For example, people with a high risk of heart disease can be provided with advertisements and have access to health information for cholesterol lowering drugs, hypertension, etc. The pharmaceutical companies and other advertisers preferably will not have direct access to the patients or their medical information. All medical information stored on the site can be de-identified to protect the privacy of patients.

The system can also provide a social network that allows users to connect with other patients of a particular phenotype and/or genotype.

It should be noted that the term “user” and the term “patient” are used somewhat interchangeably herein. The system and the method of the present invention can be operated or practiced by a user of the system and method. That user may also be a patient, and may specifically be the patient from whom the health and medical information is obtained, and for whom the output of the system is generated. However, a patient may delegate actual interaction with the system and method to a user that is acting on behalf of the patient. As such, the information relating to health may be specific to the patient, but login information may be specific to a user. Likewise, a user that is also the patient may operate or practice the system and method of the present invention, or a user that is not also the patient may operate or practice the system and method of the present invention, as would be understood by those of ordinary skill in the art.

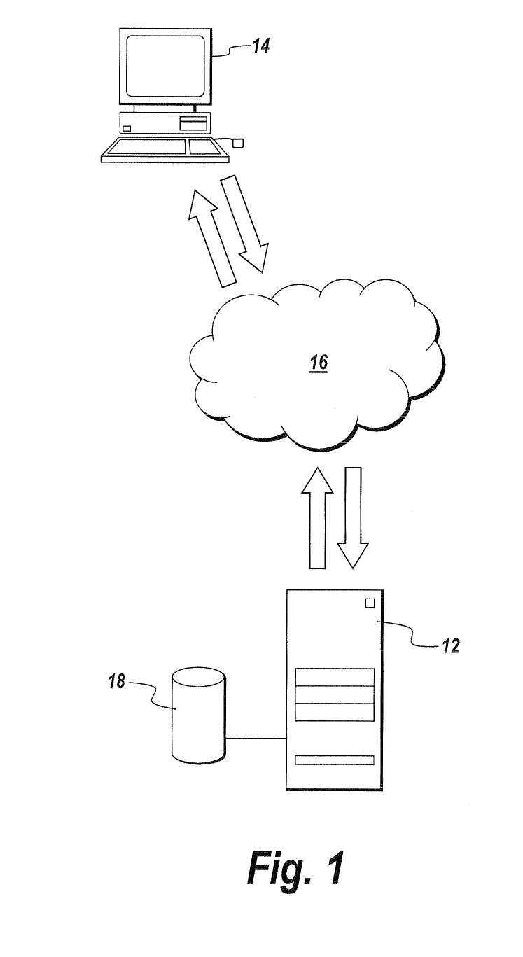

FIG. 1 schematically illustrates a representative networked computer environment in which a system for providing personalized medical health information to users can be implemented. In general, the system includes a server system 12 for delivering content to a plurality of user terminals or client devices 14 operated by users over a network 16. Each user client device 14 has an associated display device for displaying the delivered content. Each client device 14 also has one or more user input devices that enable the user to interact with a user interface on the client device 14. Input devices can include, but are not limited to touch screens, keyboards, and mice or other pointer devices.

The network 16 can take the form of a computer network such as, e.g., the Internet (particularly the World Wide Web), Intranets, or other networks. Communication to, from, and through the network 16 can occur through a hardwire connection, a network connection, or a wireless connection, including a cellular or WiFi connection, and as such further includes at least one internal antenna and/or input/output jack (not shown). The server system 12 can comprise, e.g., a Web server. The client device 14 can comprise, e.g., a personal computer or a portable communication device such as a personal digital assistant (PDA) or a cellular telephone. The client device 14 can include a browser, which may, e.g., be any of a variety of conventional web browsers.

The server system 12 includes access to one or more databases or electronic storage systems 18, which can be used to store information on system users, including patient phenotype and/or genotype information, as well as the content (including information on medical health topics and advertisements) to be delivered to client devices operated by the users.

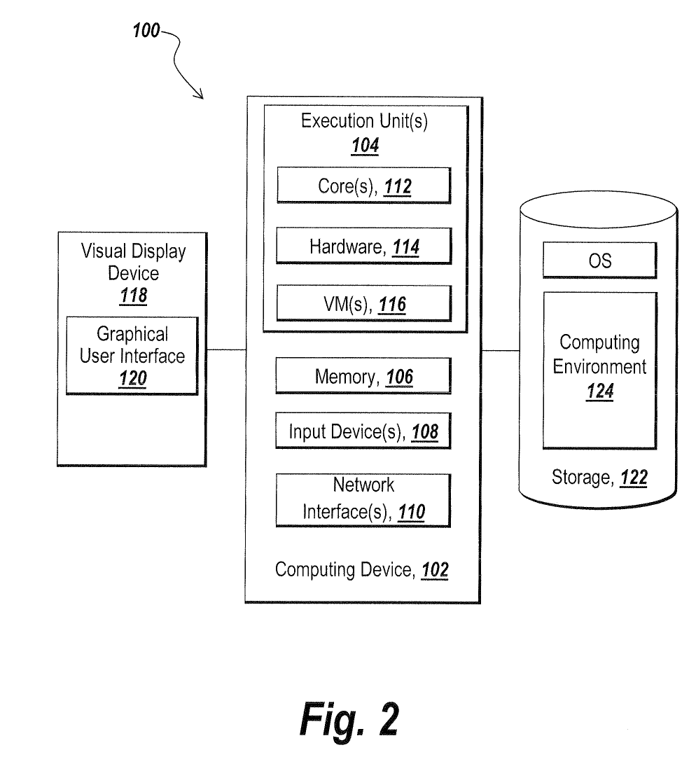

As described herein, the system and method of the present invention are implemented in a computer networked environment. Each computing component involved in the computer networked environment, including the client device 14 and the server system 12, can each take the form of its own computing environment 100. FIG. 2 depicts an example computing environment 100 suitable for practicing exemplary embodiments of the present invention. As indicated herein, the present system and method can be implemented on one or more computing devices 102. The computing environment 100 includes the computing device 102, which may include execution units 104, memory 106, input device(s) 108, and network interface(s) 110. The execution units 104 may include hardware or software based logic to execute instructions on behalf of the computing device 102. For example, depending on specific implementation requirements, execution units 104 may include: one or more processors, such as a microprocessor; single or multiple cores 112 for executing software stored in the memory 106, or other programs for controlling the computing device 102; hardware 114, such as a digital signal processor (DSP), a graphics processing unit (GPU), a field programmable gate array (FPGA), an application specific integrated circuit (ASIC), etc., on which at least a part of applications can be executed; and/or a virtual machine (VM) 116 for executing the code loaded in the memory 106 (multiple VMs 116 may be resident on a single execution unit 104).

Depending on specific implementation requirements, the memory 106 may include a computer system memory or random access memory (RAM), such as dynamic RAM (DRAM), static RAM (SRAM), extended data out RAM (EDO RAM), etc. The memory 106 may include other types of memory as well, or combinations thereof. A user may interact with the computing device 102 through a visual display device 118, such as a computer monitor, which may include a graphical user interface (GUI) 120. The computing device 102 may include other I/O devices, such as a keyboard, and a pointing device (for example, a mouse) for receiving input from a user. Optionally, the keyboard and the pointing device may be connected to the visual display device 118. The computing device 102 may include other suitable conventional I/O peripherals. Moreover, depending on particular implementation requirements of the present invention, the computing device 102 may be any computer system such as a workstation, desktop computer, server, laptop, handheld computer or other appropriate form of computing or telecommunications device that is capable of communication and that has sufficient processor power and memory capacity to perform the operations described herein.

Additionally, the computing device 102 may include interfaces, such as the network interface 110, to interface to a Local Area Network (LAN), Wide Area Network (WAN), a cellular network, the Internet, or another network, through a variety of connections including, but not limited to, standard telephone lines, LAN or WAN links (e.g., T1, T3, 56 kb, X.25), broadband connections (e.g., integrated services digital network (ISDN), Frame Relay, asynchronous transfer mode (ATM), synchronous transfer mode (STM), wireless connections (e.g., 802.11), high-speed interconnects (e.g., InfiniBand, gigabit Ethernet, Myrinet) or some combination of any or all of the above as appropriate for a particular embodiment of the present invention. The network interface 110 may include a built-in network adapter, network interface card, personal computer memory card international association (PCMCIA) network card, card bus network adapter, wireless network adapter, universal serial bus (USB) network adapter, modem or any other device suitable for interfacing the computing device 102 to any type of network capable of communication and performing the operations described herein.

The computing device 102 may further include a storage device 122, such as a hard-drive, flash-drive, or CD-ROM, for storing an operating system (OS) and for storing application software programs, such as the computing application or environment 124. The computing environment 124 may run on any operating system such as any of the versions of the conventional operating systems, any embedded operating system, any real-time operating system, any open source operating system, any proprietary operating system, any operating systems for mobile computing devices, or any other operating system capable of running on the computing device and performing the operations described herein. Furthermore, the operating system and the computing environment 124 may in some instances be run from a bootable CD.

One of ordinary skill in the art will appreciate that the above description concerning the computing environment 100 and computing device 102 is intended to encompass all conventional computing systems suitable for carrying out methods of the present invention. As such, any variations or equivalents thereof that are likewise suitable for carrying out the methods of the present invention are likewise intended to be included in the computing environment 100 described herein. Furthermore, to the extent there are any specific embodiments or variations on the computing environment 100 that are not suitable for, or would make inoperable, the implementation of the present invention, such embodiments or variations are not intended for use with the present invention.

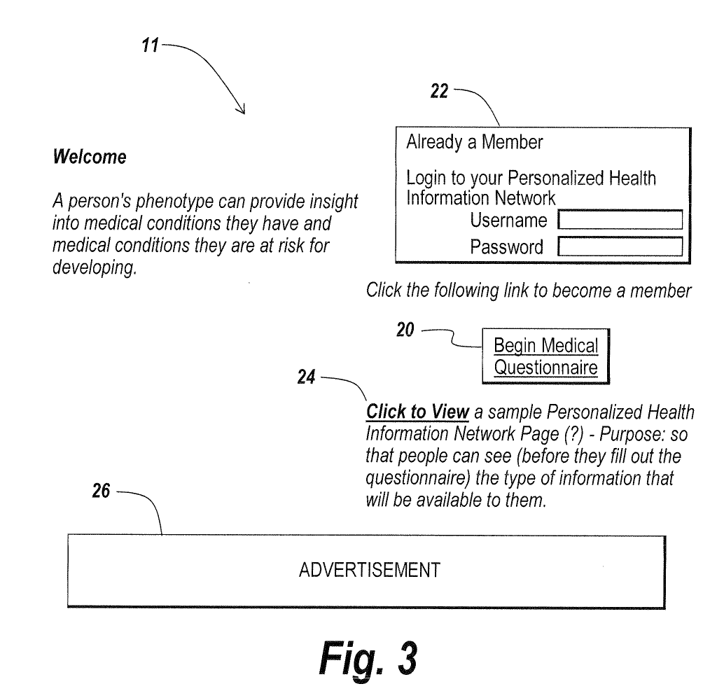

FIG. 3 illustrates a homepage starting screen 11, as would be understood by those of ordinary skill in the art, which can display to users the system of the present invention on their client devices 14. If the user is a new user, he or she can select a link to establish an account with the system and fill in a medical questionnaire to develop a phenotype. Existing users of the system can log in by entering unique user identification information, e.g., a username and password in a login block 22.

The user can also select a link 24 to view a sample personalized health management portal so that he or she can obtain a better understanding of the type of information that will be available if he or she registers to use the service.

To become a member (i.e., a registered user), a new user can be asked to enter information for creating a login, beginning by clicking on a new member link 20. Users are preferably not asked to provide personal identification information. The user can be requested to enter the following: email address, username, password, security question, type in a word appearing in a box (to restrict access to the system by automated programs), agreed to acceptance of terms & conditions. Thereafter, the user is routed to the beginning of a medical questionnaire, an example of which is attached as Appendix “A”.

The system can include multiple tiers of advertisements, depending on the particular webpage accessed by the user. For example, Tier (I) advertisements can be provided on the site Homepage in an advertising block 26, where the highest traffic can be expected.

Tier (II) advertising can be provided on the homepage of a user's personalized health management portal, or other subsequent pages within the system. This page has the highest traffic for each “username,” i.e., each registered user can be expected to access this page more frequently than any other page on the site.

Tier (III) advertising can be provided on the pages addressing specific medical conditions within a user's personalized health management portal. This page can be expected to have the highest traffic for each “username” with the specific “condition.”

Tier (III+) advertising can comprise additional tiers of advertising for further sub pages accessed by users.

To encourage return user traffic to the system, when new medical information becomes available for a user or patient, his or her personalized health management portal can be automatically updated, a user can be sent an email (or otherwise notified) informing him or her that new medical information is available. A link to the system website can be included in the email for convenience.

After a user completes the Medical History Questionnaire, he or she can log into their personalized health management portal Homepage using their Username and Password. Information entered by the user in the medical questionnaire is used to determine the patient's phenotype. Based on that phenotype, personalized health information can be provided to the user. More specifically, the health management portal tool of the present invention takes all of the information provided concerning a patient and implements a process wherein a number of analyses and operations are conducted to result in an indication of present medical conditions and risks for future medical conditions. The system and method of the present invention transform the information concerning the patient into the output discussed herein. The methodology involves generating a predictive model. The predictive model or models make use of a one or more algorithms executing on at least one processor of a computing apparatus. The predictive model or models determine and indicate or communicate a potential for development by the patient of disease and adverse health conditions.

The steps for implementation of the present invention, in operation, are as follows . . . . The data is first provided to the system (step 100). The data can include phenotype data specific to a patient, biometric data specific to the patient, medical claims data specific to the patient, and organizational data specific to an organization to which the patient belongs. Phenotype data can include data fields comprising height, weight, waist circumference, biometric data, smoking frequency, alcohol consumption, lifestyle data, emotional data, behavioral data, and the like. Biometric data can include data fields comprising total cholesterol, HDL cholesterol, LDL cholesterol, triglycerides, fasting glucose, hemoglobin A1c, ALT liver enzyme, C-Reactive Protein, Complete Blood Count, and the like. Medical claims data can include data fields comprising health insurance claims for medical procedures, prescription medication cost, doctor visit fees, and the like. Organizational data can include data fields comprising current health by condition, health risks by condition, productivity, absenteeism, lost time, predictive modeling, medical claims analysis, program eligibility, program participation, direct medical cost analysis, indirect medical cost analysis, return on investment, and the like. One of ordinary skill in the art will appreciate that the above data fields are all illustrative of what may be utilized in a health risk predictive model. Accordingly, the present invention is by no means limited to the precise data fields provided herein, which are provided merely for illustrative purposes.

The health management portal tool of the present invention takes whatever data is provided and executes a number of different algorithms to assess the potential for development by the patient of disease and adverse health conditions. The algorithms being used to phenotype individuals can include: hypertension (high blood pressure), high cholesterol, coronary heart disease, osteoporosis, diabetes, visceral fat, metabolic syndrome, gum disease, and depression.

### Hypertension:

Individuals are classified as either at-risk or not-at-risk for hypertension. If an individual has systolic >=130 or diastolic >=85 then they are identified as being at risk for hypertension. For an individual at risk, they are further classified for the severity of their risk according to the highest category of either the systolic or diastolic value:

| | Systolic Blood | |
|---|---|---|
| Category | Pressure | Diastolic Blood Pressure |
| Low | 130-139 | 85-89 |
| Medium | 140-159 | 90-99 |
| High | 160-209 | 100-119 |
| Extreme (See your doctor | >=210 | >=120 |
| immediately) | | |

See Joint National Committee on Detection, Evaluation and Treatment of High Blood Pressure. The fifth report of the Joint National Committee on detection, evaluation, and treatment of high blood pressure (JNC V). Arch Intern Med. 1993; 153: 154-183.

### Cholesterol:

The algorithm used to determine high cholesterol is similar to hypertension in that an individual is classified as at-risk or not-at-risk. An individual may be at risk if they meet any of the following criteria:

| | |
|---|---|
| Total Cholesterol | >=200 |
| LDL Cholesterol | >=130 |

The severity of cholesterol levels may be based on the following:

| Severity | Total Cholesterol Level |
|---|---|
| Medium | >=200 and <240 |
| High | >=240 |

See Bachorik P S, Ross J W. National cholesterol education program recommendations for measurement of low-density lipoprotein cholesterol: Executive Summary. Clin Chem. 1995; 41: 1414-1420. See also Jacobs D, Demott W, et al. Laboratory Test Handbook, 3rd edition. Lexi-Comp Inc. Hudson, Ohio, 1994, page 250. Jacobs D, Demott W, et al. Laboratory Test Handbook, 4th edition. Lexi-Comp Inc. Hudson, Ohio, 1996, pages 110-111.

Coronary Heart Disease: The Framingham 10 year risk algorithm can be used as an indicator for Coronary Heart Disease.

The risk levels may be stratified according to:

| | Coronary Heart Disease Risk Percent (10 |
|---|---|
| Severity | year) |
| Not shown | <10% |
| Low | 10-20% |
| Medium | 20-30% |
| High | 30+% |

See Framingham Circulation 1998; 97:1837-1847

### Osteoporosis:

The FRAX model may be used to determine risk for Osteoporosis.

| | Major Osteoporotic Fracture Percent (10 |
|---|---|
| Severity shown to user | year) |
| Not shown | <10% |
| Low | 10-20% |
| Medium | 20-30% |
| High | 30+% |

### REFERENCES

FRAX Osteoporosis International 2008; 19:385-97

### Diabetes:

The tool may implement the algorithm noted in the below noted references.

After identifying a user's risk percent, the tool may show the following risk level:

| Severity shown to user | Probability of Type 2 Diabetes |
|---|---|
| Not shown | <10% |
| Low | 10-20% |
| Medium | 20-30% |
| High | 30+% |

See Griffin S J, Little P S, et al. Diabetes risk score: towards earlier detection of Type 2 diabetes in general practice. Diabetes Metabolism Research and Reviews. 2000; 16: 164-171. See also Park P J, Griffin S J, et al. The performance of a risk score in predicting undiagnosed hyperglycemia. Diabetes Care. 2002; 25: 984-988. See also Spijkerman A M W, Yuun M F, et al. The performance of a risk score as a screening test for undiagnosed hyperglycemia in ethnic minority groups. Diabetes Care. 2004; 27: 116-122.

### Visceral Fat:

The amount of visceral fat in an individual is determined by the following algorithms:

First visceral fat (in cm²) is calculated

For Caucasian men:

x=(waist size(cm)*0.03)+(age*0.02)+0.32+0.66

For non-Caucasian men:

x=(waist size(cm)*0.03)+(age*0.02)+0.66

For Caucasian women:

x=(BMI*0.03)+(waist size(cm)*0.02)+(age*0.02)+0.2+1.13

For non-Caucasian women:

x=(BMI*0.03)+(waist size(cm)*0.02)+(age*0.02)+1.13

### For all:

vFat(cm²)=exp(x)

After calculating visceral fat, individuals may be put into relative quintiles compared to US average and then assigned a risk category. This risk category may be either classified as “normal weight obesity” (<30 bmi) or “excess abdominal fat” (>=30 bmi):

Females

| | | Risk of Normal Weight |
|---|---|---|
| Quintile | Amount of vFat | Obesity |
| Top 20% | <67.6 cm² | None |
| Top 40% | <105.3 cm² | Low |
| Top 60% | <140.1 cm² | Medium |
| Top 80% | <193.6 cm² | High |
| Top 100% | >=193.6 cm² | Urgent |

Males

| | | Risk of Normal Weight |
|---|---|---|
| Quintile | Amount of vFat | Obesity |
| Top 20% | <106.3 cm² | None |
| Top 40% | <141.3 cm² | Low |
| Top 60% | <189.5 cm² | Medium |
| Top 80% | <244.6 cm² | High |
| Top 100% | >=244.6 cm² | Urgent |

See Schreiner, P J, et al. Sex-specific Associations of Magnetic Resonance imaging-derived Intra-abdominal and Subcutaneous Fat Areas with Conventional Anthropometric Indices. American Journal of Epidemiology. Vol. 144, No. 4. 1996. See also Heritage Study Field Validation.

### Metabolic Syndrome:

Metabolic syndrome may be assessed in individuals to predict their risk of developing cardiovascular disease and type 2 diabetes. See Pischon, T., et al. Inflammation, the metabolic syndrome, and risk of coronary heart disease in women and men. Atherosclerosis Vol. 197.392-399. 2008.

### Depression:

The Patient Health Questionnaire (PHQ)-2 and the PHQ-9 may be implemented to assess depression and other mental health conditions within an individual. See PHQ-2: Thibault J M, Prasaad Steiner, R W. Efficient identification of adults with depression and dementia. American Family Physician, Vol. 70/No. 6 (Sep. 15, 2004). See also PI-IQ-9: Spitzer, R., Williams, B. W., Kroenke, K., et al. PHQ-9. PRIME MD TODAY, Pfizer, Inc. 2009.

The system and method of the present invention can predict risk of unfavorable health conditions or events using one or more of the above algorithms. For example, the system and method of the present invention can predict the risk of a heart attack from known risk factors using the Framingham risk algorithm. Additionally, the system and method of the present invention can predict the risk of a heart attack from the predicted risk of diabetes and/or amount of visceral fat.

One of ordinary skill in the art will appreciate that the above listing and description of various models and algorithms is merely illustrative of the type of algorithms that may be utilized in conjunction with the present invention to provide the desired analysis and outcome. As such models are improved or even replaced with different models over time, to the extent such models can be reduced to an executable algorithm, such models are anticipated for use in conjunction with the system and method of the present invention. As such, the present invention is by no means limited to the specific algorithms provided herein. Such models and algorithms are merely provided to demonstrate actual algorithms that may be implemented together with the system and method of the present invention.

The system and method of the present invention outputs the potential for development by the patient of disease and adverse health conditions. Such disease and adverse health conditions can include Heart disease, High Blood Pressure, High Cholesterol, Diabetes, Osteoporosis, Metabolic Syndrome, Gum Disease, Body Fat Composition, Chronic Obstructive Pulmonary Disease, Depression, mental disorders, and the like. Again, one of skill in the art will appreciate that there can be numerous potential diseases and adverse health conditions, and that the above are merely illustrative. As such, the present invention is by no means limited to the specific list of potential disease and conditions, but is intended to have the capability to assess risk of all known diseases and conditions that can be determined using an algorithmic based analysis.

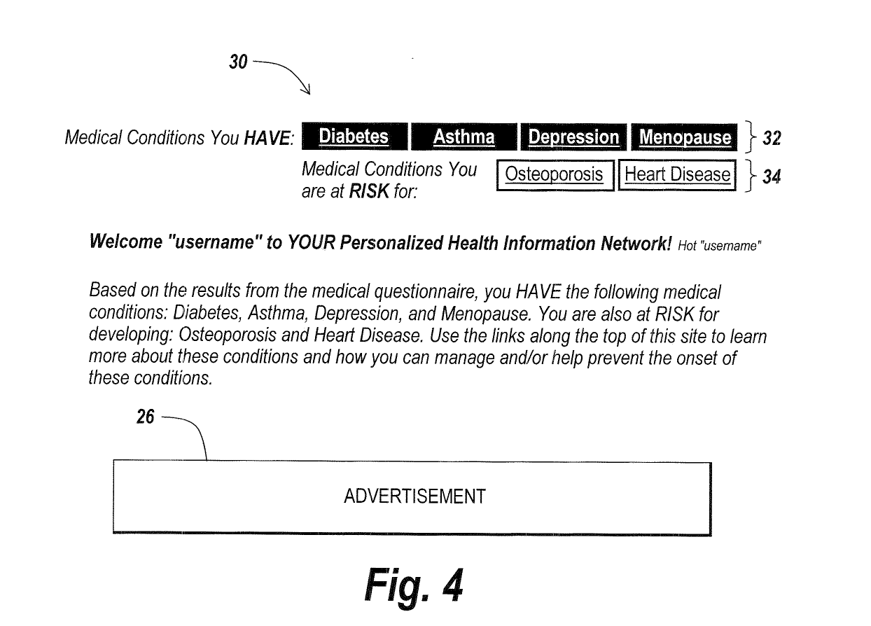

FIG. 4 is an example screenshot 30 of a webpage providing personalized information for a patient whose phenotype has been determined.

The screenshot 30 of a webpage providing personalized information can serve as a homepage for each user's personalized health management portal, identified by a Tag Line within the Header Bar personalized for each user, i.e., “Username's” personalized health management portal. A Welcome Message expressing the “username” can be shown to confirm the correct user.

The webpage identifies one or more medical conditions that the patient has (in this example, diabetes, asthma, depression, and menopause) at a medical condition indication 32. The webpage also identifies medical conditions that the patient is at risk for (in this example, osteoporosis and heart disease) at a medical condition risk indication 34. Links can be provided for each of the conditions to other subpages providing additional information on each of the conditions.

The user can access the Medical Questionnaire through the Homepage for their personalized health management portal to update their information. Their Homepage can be updated automatically with health information, ads, etc. specific to their phenotype.

One or more advertisements can be provided in the advertising block 26. Advertisements could include streaming commercials and/or static advertisements for pharmaceuticals, medical devices, hospitals, doctors, insurance, nutrition, etc. Advertisements and medical information on a user's homepage is preferably related to any of the medical conditions that the patient either has or is at risk for developing.

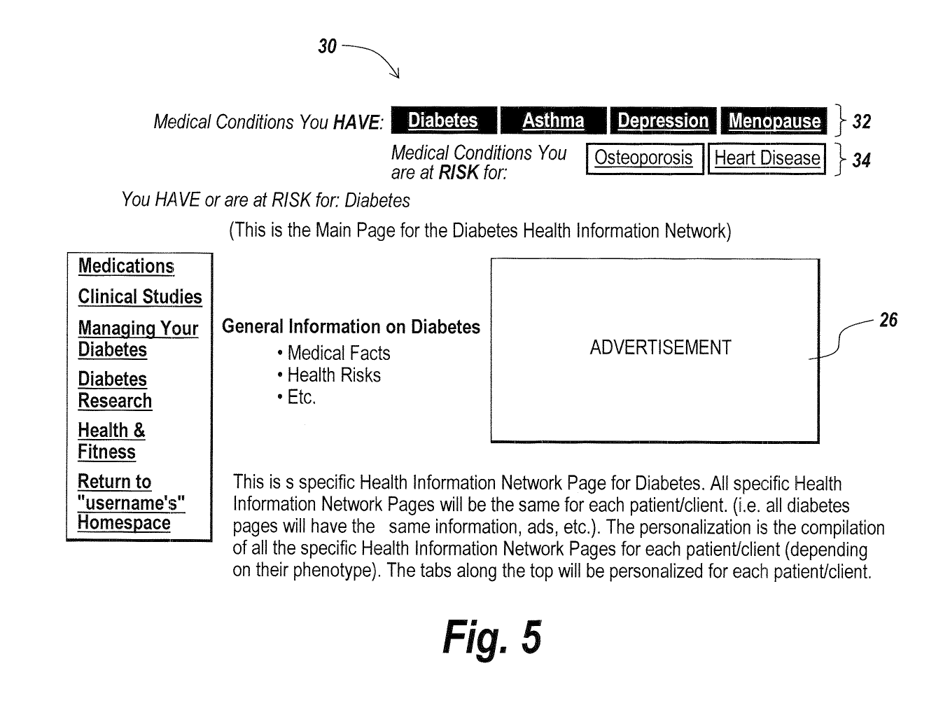

FIG. 5 is a screenshot 40 of an example—personalized health management portal—condition Homepage, which can be accessed by the user or by selecting one of the medical condition indications 32, 34 shown in FIG. 4.

In the illustrative example, a homepage description for Diabetes is provided. The homepage for each condition contains general health information for that specific condition. Links can be provided along the left side of the page to sub-pages that contain more detailed information (including medical information, advertisements, etc.) for the condition.

In addition, the condition homepage can include Tier (III) advertisements. These advertisements can be specific to the subject condition (diabetes in this example). The advertisements can include medication advertisements, light commercial advertisements, medical device advertisements (such as blood monitors), and advertisements on nutrition, fitness, and treatments.

Tier (III+) Advertising space may be displayed on the subpages of the condition. These advertisements are preferably related to the subject matter of the subpages.

In accordance with one or more alternative embodiments, in addition to phenotyping, blood samples can be collected from consenting patients (e.g., in a PhenotypingIT blood collection facility or through a mobile van, mall site, or through collaboration with a commercial entity such as Quest Diagnostics, CVS or other pharmacies, clinics, Home Access, or the like). Biometric screening as well as the patient's genotype can be determined from the blood sample. This can be used to provide additional, personalized health care information relating to biometric and genetic results. Advantageously, the phenotyping information available on patients can be uniquely correlated with the genotyping data. For example, if patients have symptoms of memory loss and precursor genes for Alzheimer's, they are at greater risk of developing Alzheimer's disease. If they have a family history of breast cancer and the BRAC gene, they are more likely to get this disease. New information is becoming frequently available for diabetes risk genes. This disease is polygenetic, so each gene contributes a little to the overall disease risk, and those at higher weight, with a larger waist circumference, with a family history and with a greater risk determined by PA genotyping are at greater overall clinical risk for the disease. This uniquely personalized information can be made available to patients whose genotype and phenotype are known by the system.

In addition, users will have the opportunity to confidentially participate in an online health care community. This participation can be based not only on actual disease but also on unique phenotype information users provide. For example, a patient with an above normal weight and waist circumference is at risk for diabetes, and he or she can participate in such a community to learn ways to prevent the disease or its manifestations, or of uniquely available resources in a geographic community, etc. Furthermore, participation in a community could be based on genotype information and/or genotype/phenotype correlations.

The one or more databases store user and patient information, including user logon/ID information (e.g., e-mail address) and medical information including the answers to the Medical Questionnaire and the calculated risks for Medical Conditions. The answers and risks can be classified several ways within database (e.g., according to the medical condition, medications, medical conditions within specific demographics; such as age, gender, race, level of education, etc., and geographic location). Each condition is linked to corresponding medical information, advertisements, etc.

The content displayed to the users can be categorized by medical condition and a tiered advertisement payment plan. Other methods of categorization are also possible.

In accordance with some embodiments of the present invention, there are two databases: one database containing patient medical questionnaire information and calculated risks, and the other database containing content to be displayed on the website including medical information and ads, with the content being linked to particular patients and/or medical conditions. Alternatively, a single central database can be used containing both the user information as well as the content.

In addition to the collection of patient information, and the automated ability to execute one or more algorithms for assessing or predicting risk of unfavorable health conditions or events, the present invention further executes one or more automated processes to assess results of each individual health assessment algorithm, and determine an overall potential for development by the patient of disease and adverse health conditions.

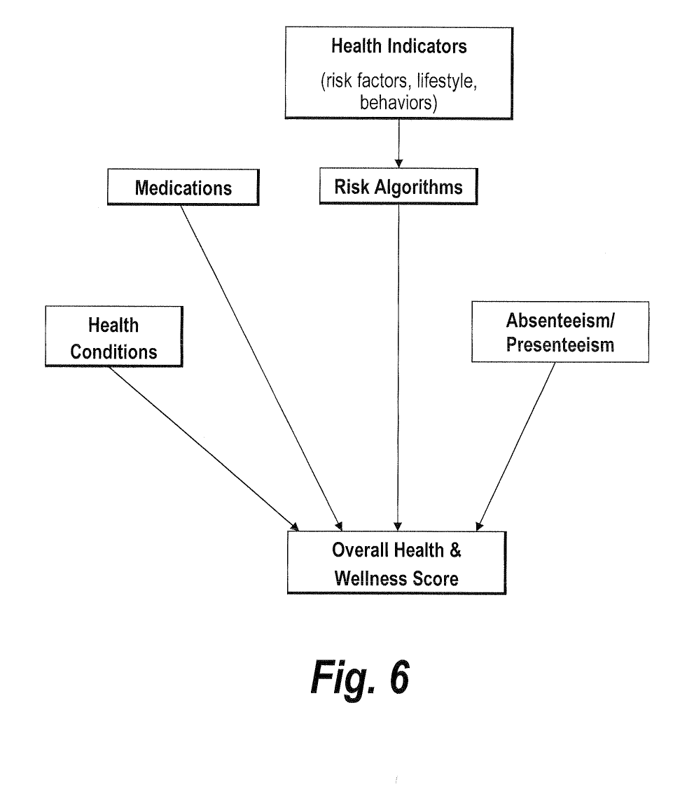

In accordance with one example embodiment of the present invention, and looking at FIG. 6, an overall potential for development by the patient of disease and adverse health conditions can be determined by considering a number of different factors. For example, predictive algorithms can be executed to determine existing adverse health conditions, medications, health indicators (e.g., risk factors, lifestyle, behaviors, and the like), and daily organizational behavior (e.g., absenteeism, presenteeism, or other trackable organizational behaviors) as discussed elsewhere herein. One or more further predictive algorithms can be executed to determine an overall potential for development by the patient of disease and adverse health conditions. Such further predictive algorithms can take the results of the individual predictive algorithms, assess the results in a more holistic approach, and indicate a potential for development by the patient of disease and adverse health conditions taken altogether rather than as separate and individual assessments for each type of condition.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

The techniques described above are preferably implemented in software, and accordingly one of the preferred implementations of the invention is as a set of instructions (program code) in a code module resident in the random access memory of a computer or computing device 102 as described herein. Until required by the computer, the set of instructions may be stored in another computer memory, e.g., in a hard disk drive, or in a removable memory such as an optical disk (for eventual use in a CD or DVD ROM) or floppy disk (for eventual use in a floppy disk drive), a removable storage device (e.g., external hard drive, memory card, or flash drive), or downloaded via the Internet or some other computer network. In addition, although the various methods described are conveniently implemented in a computer selectively activated or reconfigured by software, one of ordinary skill in the art would also recognize that such methods may be carried out in hardware, in firmware, or in more specialized apparatus constructed to perform the specified method steps.

Continuing discussion of the present invention, and in accordance with one embodiment of the present invention, a computer implemented software-based product and service are provided that assess the health and wellness of members within an organization, group, and/or entity. The organization, group, and/or entity can be, for example, employees of a company, members of a health care organization or plan, or the like. Alternatively, the organization, group, and/or entity can be defined as broadly as any user of the software-based product, and each user could contribute to the organization, group, and/or entity of all users, regardless of any physical or virtual locations or affiliations. In conjunction with this assessment, customized health and wellness information can be determined. In addition, reports detailing and summarizing aggregated findings can be generated for purposes including reduction of health care costs.

The present invention includes a data entry component for executing a health risk assessment (HRA), a health management portal tool for each user to access information and generate analyses and reports, and the option for template or pre-designed customer reports based on the data, optionally using a reporting portal tool.

The health risk assessment (HRA) is to be completed by users, such as employees and/or members within an organization, or other users, preferably in an online environment. The HRA asks questions relative to the leading causes of heightened direct and indirect health care costs, including leading causes of absenteeism and presenteeism. The HRA also asks questions whose answers aid in determining the phenotype of the patient, including analysis of subcutaneous and visceral body fat composition, and metabolic syndrome.

In accordance with one example embodiment, the HRA can be multi-paged and each page can be organized into a multi-column format, for example. One column can outline the organization of the HRA and the status of completion. One column can contain the questions. One column can provide real-time feedback to the user as they are taking the HRA. The feedback can include: real-time health risk assessment, tips, explanation of the questions, coaching relevant to the HRA, and the like, and may include additional features not specifically listed here. The HRA is dynamic in that specific questions are asked depending on demographics, lifestyle, etc. A Summary of the HRA can be automatically generated for each user at the completion of the HRA. The Summary can be organized in an interactive dashboard revealing to the user information on their health risks and risk factors. The user may view, save, and print the HRA Summary. The present invention may store a patient's information in a Personal Health Record (PHR) that the user can view and update. The user may have the option to sync their PHR with the Google® Health platform, or other similar platforms. The user can view and update their HRA and PHR using the health management portal tool.

A reporting portal tool provided herewith serves as a risk assessment, analysis, and management tool to allow the organization, group, and/or entity to assess the indirect and direct costs based on the overall health and wellness, absenteeism rates, and presenteeism rates of their members and/or employees. The health management portal tool and the reporting portal tool additionally serve all users of the system, both for information input, and information output actions.

The portal tools can be provided to employers, health plan providers, employees/members, brokers, and other value added resellers, for example. The portal tools can be additionally provided to users entering the medical information into the system, and generating, and viewing desired reports and analyses.

The health management portal tool, having aspects of the personalized health management portal referred to above can be automatically generated for each user that completes an HRA. The health management portal tool may provide access to health and wellness information, including information directly related to their current health conditions and conditions that the patient may be at risk for developing. The health and wellness information may come from several resources, including but not limited to, content generated within the database and system of the present invention, or from content originating form 3rd party providers. The health management portal tool may include, but is not limited to, interactive tools for tracking and managing health and wellness over time.

Customer reports can be generated for the appropriate person(s) in charge of an organization, group, or other entity. All reports may be developed by the system and method of the present invention, using aggregated data. No personal identifying information is required to be disclosed to any organization, group, or other entity. Reports include, but are not limited to: results from the HRA, Percentage of Employees and/or Members with Specific Medical Conditions, Performance Reports, Health Care Cost Analysis, Comparative Studies of Performance Reports versus Medical Claims Activity, Comparative Reports across the National Average, Comparative Reports across the Industrial Average, Data Trending & Predicting, Health Care Cost Prediction based on Health Risks of Employees and/or Members. Administrators of an organization, group, or other entity, may have the ability to generate custom reports from aggregated data using data filtering tools within the reporting portal.

There are multiple options for displaying to users, including the appropriate person(s) in charge of an organization, group, and/or entity, of the system and method of the present invention the various reports as described herein. For example, reports can be viewed using the reporting portal tool through an on-line dashboard (password protected), reports can be accessed, viewed, and printed. A screen shot of the dashboard may be printed or saved. Hard copy reports may be published, or generated and forwarded to select personnel.

The system and method of the present invention reduces direct and indirect health care costs. The HRA completed by users collects valuable and useful information. Custom reports can be generated based on the information entered by users, both for organization, group, or entity purposes, as well as for individual user purposes.

Health Plan Providers can use the system and method of the present invention, within their organization and distribute to their members. Alternatively, Providers can distribute client organizations. As a risk assessment, analysis, and management tool for Health Plan Providers, the present invention can reduce claims payout, perform member health assessments, generate custom reports, and provide benefits to users, such as access to the portal tools.

Value Added Resellers and brokers can make use of the system and method of the present invention as a platform to launch a comprehensive wellness program, or enhance preexisting programs. Again, the HRA can be useful to collect and assess medical information. Custom reports can be generated, and users that are customers can be provided with the portal tools.

The system and method of the present invention can be used to assess, analyze, and manage the health and wellness of patients, including family members of employees and/or members. The service can be provided as a Subscription as a Service (SaaS) Model. The system and method of the present invention can be built using a Portal Server, allowing for a customer and user to customize the user interface for the portals.

The system and method of the present invention include methods of entering HRA, PHR and other Medical History Information, such as filling out the HRA online. Alternatively the PHR and HRA information can be uploaded to and from 3rd party sources, such as, Google® Health, Microsoft® Health Vault, and the like.

The health management portal tool is capable of automating algorithms for determining health risks. The automation process includes analyzing self-reported data, integrating with and analyzing biometric data, medical claims data, and organizational data. Additional risk prediction (above what competitors predict) includes estimating the levels of visceral (intra-abdominal) fat and Metabolic Syndrome; each which puts a person at elevated risk of developing certain health conditions.

The system of the present invention can include Social Networking features targeted to help Users manage current health conditions as well as manage health risks. Social Networking features can be internally generated, or be integrated with 3rd party forums, blogs, discussion boards, challenges, support groups, and the like.

The system can include advertisements targeted to users who are at risk for certain health conditions in addition to advertisements targeted to users who have certain health conditions.

The system can generate and distribute lists of users to 3rd parties, including but not limited to health coaches, councilors, nurses, doctors, etc. Integrating with these 3rd parties can enhance the effectiveness of the program by targeting care to specified users, depending on level of care and support needed.

The system can integrate with a multi-channel communication platform. Modes of communication can include but are not limited to direct mail, email, sms/text, and voice messages. Communications/notifications can be event triggered based on risk levels and/or predicted risks. Communications/notifications can also be used to transmit information surrounding the functions of the System, including initiating wellness programs, inviting Users to participate, targeting health information, reminders to view health reports, etc. Communication via the System can be transmitted and received between Employers, Members/Users/Employees, Payers, Brokers, Consultants, Coaches, and the like.

Upon creation of the database containing the patient medical information, additional services can be implemented. For example, testing of known SNPs that cause condition (i.e. if phenotype shows risk for developing osteoporosis, offer option to get genetically tested for the known osteoporosis genes can be performed, perhaps also making use of a saliva testing kit).

The system and method of the present invention informs organizations, groups, and/or entities of health problems facing its users/members, as well as a prevalence thereof. Impact of such health problems on the organization, group, or entity, can be determined, including work performance, sickness absence, industrial accidents, and disability. From that, monetary values can be placed on such impacts.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention.

What is claimed is:

1. A method, comprising:
receiving patient data comprising one or more of phenotype data specific to a patient, biometric data specific to the patient, medical claims data specific to the patient, and organizational data specific to an organization to which the patient belongs;
generating one or more predictive models based on the patient data, using one or more algorithms executing on at least one processor of a computing apparatus, the one or more predictive models determining and indicating potential for development by the patient of disease and adverse health conditions; and
outputting the potential for development by the patient of disease and adverse health conditions.

2. The method of claim 1, further comprising periodically automatically updating the patient data when new information is provided.

3. The method of claim 1, further comprising periodically automatically updating the one or more predictive models and indicating the potential for development by the patient of disease and adverse health conditions by modifying the one or more algorithms.

4. The method of claim 1, wherein the phenotype data specific to the patient comprises one or more data fields of the group of data fields comprising height, weight, waist circumference, biometric data, smoking frequency, alcohol consumption, lifestyle data, emotional data, and behavioral data.

5. The method of claim 1, wherein the biometric data specific to the patient comprises one or more data fields of the group of data fields comprising total cholesterol, HDL cholesterol, LDL cholesterol, triglycerides, fasting glucose, hemoglobin A1c, ALT liver enzyme, C-Reactive Protein, and Complete Blood Count.

6. The method of claim 1, wherein the medical claims data specific to the patient comprises one or more data fields of the group of data fields comprising health insurance claims for medical procedures, prescription medication cost, and doctor visit fees.

7. The method of claim 1, wherein the organizational data specific to an organization to which the patient belongs comprises one or more data fields of the group of data fields comprising current health by condition, health risks by condition, productivity, absenteeism, lost time, predictive modeling, medical claims analysis, program eligibility, program participation, direct medical cost analysis, indirect medical cost analysis, and return on investment.

8. The method of claim 1, wherein the one or more algorithms comprise one or more algorithms of the group of algorithms comprising coronary heart disease models, blood pressure models, cholesterol models, osteoporosis models, visceral fat models, diabetes models, metabolic syndrome models, and depression models.

9. The method of claim 1, wherein outputting the potential for development by the patient of disease and adverse health conditions comprises displaying via a user interface an indication of the potential.

10. The method of claim 9, further comprising providing access through the user interface to one or more of social networks, advertisements, and electronic communication tools.

11. The method of claim 1, further comprising sending out an automatic notification when a change to data or the one or more predictive models alters the indication of the potential for development by the patient of disease and adverse health condition.

12. In a networked computer environment, a system, comprising:
a storage device storing patient data comprising one or more of phenotype data specific to a patient, biometric data specific to the patient, medical claims data specific to the patient, and organizational data specific to an organization to which the patient belongs;
at least one processor provided with executable instructions for generating one or more predictive models, using one or more algorithms executing on the at least one processor, the one or more predictive models determining and indicating a potential for development by the patient of disease and adverse health conditions; and
an output mechanism configured to output the potential for development by the patient of disease and adverse health conditions.

13. The system of claim 12, wherein the phenotype data specific to the patient comprises one or more data fields of the group of data fields comprising height, weight, waist circumference, biometric data, smoking frequency, alcohol consumption, lifestyle data, emotional data, and behavioral data.

14. The system of claim 12, wherein the biometric data specific to the patient comprises one or more data fields of the group of data fields comprising total cholesterol, HDL cholesterol, LDL cholesterol, triglycerides, fasting glucose, hemoglobin Ale, ALT liver enzyme, C-Reactive Protein, and Complete Blood Count.

15. The system of claim 12, wherein the medical claims data specific to the patient comprises one or more data fields of the group of data fields comprising health insurance claims for medical procedures, prescription medication cost, and doctor visit fees.

16. The system of claim 12, wherein the organizational data specific to an organization to which the patient belongs comprises one or more data fields of the group of data fields comprising current health by condition, health risks by condition, productivity, absenteeism, lost time, predictive modeling, medical claims analysis, program eligibility, program participation, direct medical cost analysis, indirect medical cost analysis, and return on investment.

17. The system of claim 12, wherein the one or more algorithms comprise one or more algorithms of the group of algorithms comprising coronary heart disease models, blood pressure models, cholesterol models, osteoporosis models, visceral fat models, diabetes models, metabolic syndrome models, and depression models.

18. The system of claim 12, wherein the output mechanism comprises a displayed user interface.

19. The system of claim 12, further comprising a networked communicative link to one or more of social networks, advertisements, and electronic communication tools.

20. The system of claim 12, further comprising a portal tool configured in such a way as to enable a user to generate reports based on patient data.

21. A method, comprising:
providing patient data comprising one or more of phenotype data specific to a patient, biometric data specific to the patient, medical claims data specific to the patient, and organizational data specific to an organization to which the patient belongs, to a system executing a predictive model; and
receiving an indication of a potential for development by the patient of disease and adverse health conditions generated by one or more predictive models, using one or more algorithms executing on at least one processor of a computing apparatus.
